(19) United States
(12) Patent Application Publication
(10) Pub. No.: US 2018/0267462 A1
(43) Pub. Date: Sep. 20, 2018

(54) BODY SCANNING DEVICE

(71) Applicants: Diana Serban, Saskatoon (CA); Miroslav Beserminji, Toronto (CA)

(72) Inventors: Diana Serban, Saskatoon (CA); Miroslav Beserminji, Toronto (CA)

(21) Appl. No.: 15/460,134

(22) Filed: Mar. 15, 2017

(51) Int. Cl. G03H 1/00; H04N 5/232; H04N 5/247; G03H 1/04; G03H 1/08; A61B 5/00; A61B 5/055; A61B 8/13; A61B 3/14; A61B 5/145; A61B 5/157; A61B 5/021

(52) U.S. Cl. CPC G03H 1/0005; A61B 2562/04; H04N 5/247; G03H 1/0443; G03H 1/0465; G03H 1/0891; A61B 5/0064; A61B 5/745; A61B 5/055; A61B 8/13; A61B 3/14; A61B 5/14507; A61B 5/157; A61B 5/021; G03H 2001/0088; G03H 2222/36; G03H 2222/34; A61B 2576/00; A61B 2562/0233; H04N 5/23293

(57) ABSTRACT

A system and apparatus for scanning and displaying at least a portion of a body comprises at least one scanner operable to perform a scan and output a data signal pertaining to a body. The system further includes a processing circuit operable to receive the data signal from the at least one scanner and compile an image representing the body and a display operable to receive the image representing the body from the processing circuit and display the image to a user. The apparatus includes the system located within an outer casing having an interior therein operable to receive a user wherein the body of the user is scanned and the image is presented to the user within the outer casing.

### BACKGROUND OF THE INVENTION

### 1. Field of Invention

The present invention relates generally to the field of health care diagnostic tools, and in particular to a multi-modal body scanning device which produces a three-dimensional (3D) holographic display of a user's internal organs in real-time and interprets the collected data to provide the user's current health status and to predict their future health status.

### 2. Description of Related Art

In the field of health care, a variety of non-invasive diagnostic tools are utilized to visualize internal organs and to collect data relating to the state of a patient's current health. The data collected may also be used to predict a patient's future health. Although there are many types of diagnostic tools available, such as, for example, MRI, 5D Ultrasound, bio sensors, blood pressure monitors, optical scanners and retinal scanners, currently, these diagnostic tools are available independently and primarily through health care professionals.

Other systems have been developed to display internal organs and predict future health status. Examples of such systems are described in International Publication number WO2016160509A1 to Bessette and US Patent Application Publication No. US 2013/0325493 A1 to Wong et al. Such systems however do not display in real-time, and use avatars instead of actual patient images. Additionally, they may require a series of tests in order to provide the combined health data, taking place at different times and locations, which may be time consuming and frustrating for a patient. When diagnostic tools are used at different times, the health status of the patient may vary between testing times, as well.

### SUMMARY OF THE INVENTION

According to a first embodiment of the present invention there is disclosed a system for scanning and displaying at least a portion of a body comprising at least one scanner operable to perform a scan and output a data signal pertaining to a body. The system further includes a processing circuit operable to receive the data signal from the at least one scanner and compile an image representing the body and a display operable to receive the image representing the body from the processing circuit and display the image to a user.

The at least one scanner may comprise a plurality of scanners. The image representing the body may be is compiled from the scan results from a plurality of scanners. The display may comprise a screen. The display may comprise a holographic projector. The image may be displayed to a user in real time.

The system may further comprise an outer casing having an interior therein operable to receive a user. The body may comprise a body of the user located within the interior of the casing. The display may be operable to display the image representing the body to the user within the interior of the casing.

According to a further embodiment of the present invention there is disclosed a method for scanning and displaying at least a portion of a body. The method comprises utilizing at least one scanner, performing a scan of a body and outputting from the at least one scanner, a data signal pertaining to the body. The method further comprises receiving at a processing circuit the data signal from the at least one scanner, compiling an image representing the body and displaying the image representing the body received from the processing circuit to a user.

The method further comprises utilizing a plurality of scanners. The image representing the body may be compiled from the scan results from the plurality of scanners. The image may be displayed to a user in real time.

According to a further embodiment of the present invention there is disclosed an apparatus for scanning and displaying at least a portion of a body comprising an outer casing having an interior therein operable to receive a user, and at least one scanner operable to perform a scan and output a data signal pertaining to a body of the user within the interior. The apparatus further comprises a processing circuit operable to receive the data signal from the at least one scanner and compile an image representing the body and a display operable to receive the image representing the body from the processing circuit and display the image to a user.

The at least one scanner may comprise a plurality of scanning means. The image representing the body may be compiled from the scan results from a plurality of scanners. The display may comprise a screen. The display may comprise a holographic projector. The image may be displayed to a user in real time.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention wherein similar characters of reference denote corresponding parts in each view, FIG. 1 is a cross-sectional illustration of one embodiment of a system for scanning and displaying at least a portion of a human body according to one embodiment of the present invention.

FIG. 2 is a schematic of the system of FIG. 1.

FIG. 3 is a flow chart of a method for scanning and displaying at least a portion of a human body utilizing the system of FIG. 1.

### DETAILED DESCRIPTION

Referring to FIG. 1, a system for scanning and displaying at least a portion of a human body according to a first embodiment of the invention is shown generally at 10. The system comprises a casing 50 containing a plurality of input scanners 28 and a processor 14 operable to receive input data from the plurality of input scanners 28. A user may be seated on a co-formed or integrated chair 56 within the casing 50 during operation.

The processor 14 is connected to a database 12 containing patient health data and is operable to interpret and transmit data from the input scanners 28 and the database 12 to produce a plurality of output displays 30, such as, by way of non-limiting example, 2-dimensional and/or 3-dimensional displays, utilizing a digital monitor 52 or a projection camera 54, respectively. The database 12 may be populated with data from multiple samples prior to use such that the user data may be compared thereto. The output displays 30 may be contained within the casing 50, as illustrated in FIG. 1, such that the user may view them, and may also be external to the casing 50 such that a physician or other operator may view the displays 30, as well. The displays 30 may include a portable computer, tablet 32, or the like as are commonly known, located within and/or outside of the casing 50. The images shown on the displays 30 may be in real-time, and may be manipulated through a user interface device 18, such as, by way of non-limiting example, the portable computer tablet 32.

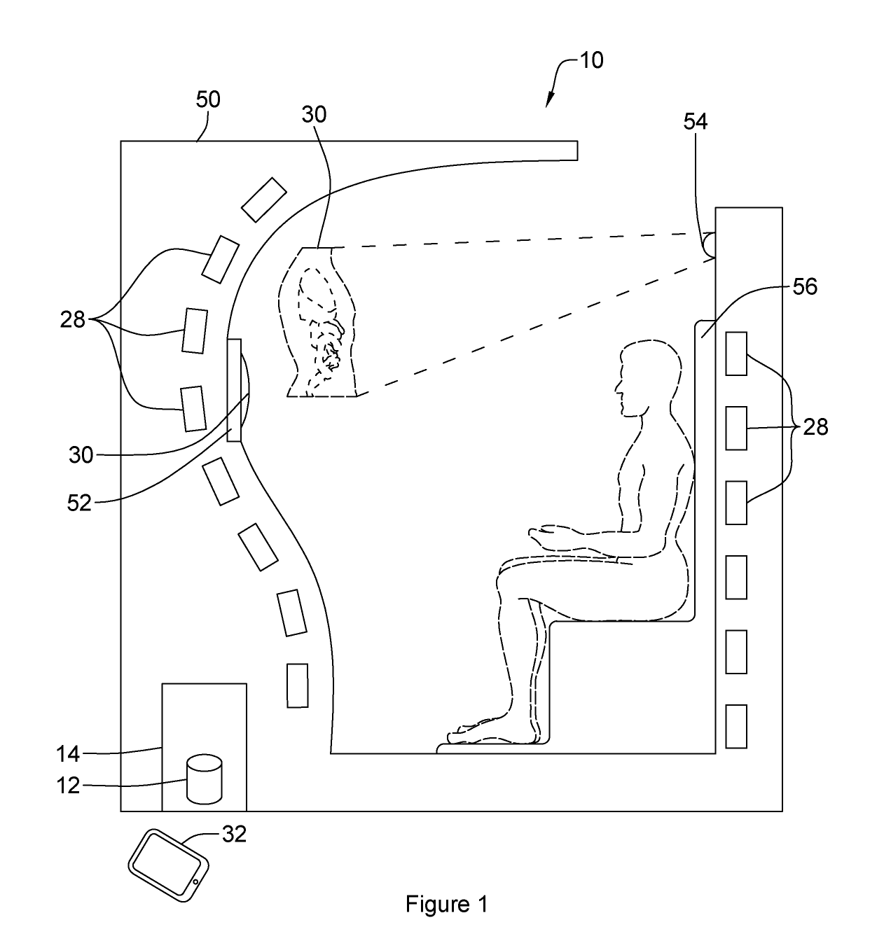

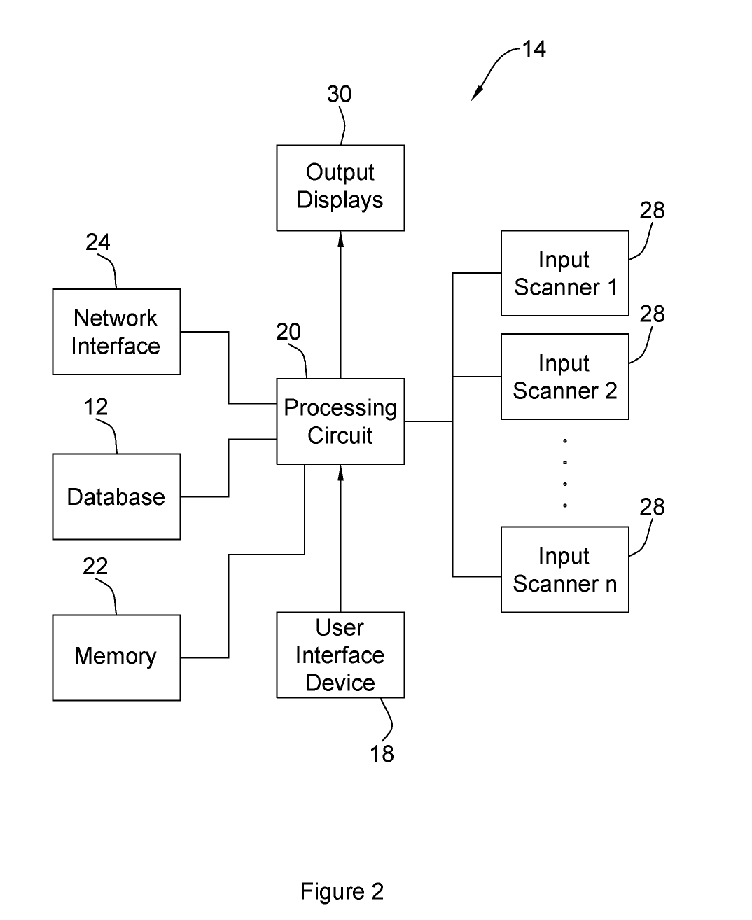

Turning now to FIG. 2, the processor 14 comprises a processing circuit 20 and memory 22 that stores machine instructions that, when executed by the processing circuit 20, cause the processing circuit 20 to perform one or more of the operations and methods described herein. As illustrated in FIG. 1, the database 12 may be contained within the processor 14, although it will be appreciated that the database 12 may be separate from the processor 14.

The processing circuit 20 may optionally contain a cache memory unit for temporary storage of instructions, data, or computer addresses. The processor 14 may include a user interface device 18, and a plurality of input scanners 28 and output displays 30 for receiving and displaying personal health data. As illustrated in FIG. 2, the processor 14 also may include a network interface 24 such as a radio transmitter, Ethernet adapter or the like for providing communication between the processing circuit 20 and external systems.

More generally, in this specification, including the claims, the term “processing circuit” is intended to broadly encompass any type of device or combination of devices capable of performing the functions described herein, including (without limitation) other types of microprocessing circuits, microcontrollers, other integrated circuits, other types of circuits or combinations of circuits, logic gates or gate arrays, or programmable devices of any sort, for example, either alone or in combination with other such devices located at the same location or remotely from each other. Additional types of processing circuit(s) will be apparent to those ordinarily skilled in the art upon review of this specification, and substitution of any such other types of processing circuit(s) is considered not to depart from the scope of the present invention as defined by the claims appended hereto. In various embodiments, the processing circuit 20 can be implemented as a single-chip, multiple chips and/or other electrical components including one or more integrated circuits and printed circuit boards.

Computer code comprising instructions for the processing circuit(s) to carry out the various embodiments, aspects, features, etc. of the present disclosure may reside in the memory 22. In various embodiments, the processing circuit 20 can be implemented as a single-chip, multiple chips and/or other electrical components including one or more integrated circuits and printed circuit boards. The processing circuit 20 together with a suitable operating system may operate to execute instructions in the form of computer code and produce and use data. By way of example and not by way of limitation, the operating system may be Windows-based, Mac-based, or Unix or Linux-based, among other suitable operating systems. Operating systems are generally well known and will not be described in further detail here.

Memory 22 may include various tangible, non-transitory computer-readable media including Read-Only Memory (ROM) and/or Random-Access Memory (RAM). As is well known in the art, ROM acts to transfer data and instructions uni-directionally to the processing circuit 20, and RAM is used typically to transfer data and instructions in a bi-directional manner. In the various embodiments disclosed herein, RAM includes computer program instructions that when executed by the processing circuit 20 cause the processing circuit 20 to execute the program instructions described in greater detail below. More generally, the term “memory” as used herein encompasses one or more storage mediums and generally provides a place to store computer code (e.g., software and/or firmware) and data that are used by the user interface device 18. It may comprise, for example, electronic, optical, magnetic, or any other storage or transmission device capable of providing the processing circuit 20 with program instructions. Memory 22 may further include a floppy disk, CD-ROM, DVD, magnetic disk, memory chip, ASIC, FPGA, EEPROM, EPROM, flash memory, optical media, or any other suitable memory from which processing circuit 20 can read instructions in computer programming languages.

The user interface device 18 may receive user input commands via touchscreen, a physical keyboard, virtual keyboard, etc. The plurality of input scanners 28 may include such as, by way of non-limiting example, MRI, ultrasound, bio sensors, Vega machine, segmental electrograph, air analyser, heat sensors, optical scan, retinal scanner, urine analyser, stool analyser, blood analyser, spinal fluid analyser, blood pressure monitor, heart monitor, etc., as are commonly known.

To perform an air analysis, it will be appreciated that doors may be provided on the casing 50 to enclose the casing 50 and seal the interior air therein.

To perform a urine or stool analysis, it will be appreciated that at least one slot as are commonly known may be provided inside or outside the casing 50 into which the samples can be inserted.

To perform a blood analysis, it will be appreciated that a blood withdrawal device, such as an ultrasound guided robotic arm such as produced by Veebot LLC may be incorporated with the chair 56 such that the user's arm would be positioned within a blood withdrawal slot or cuff.

To perform a spinal fluid analysis, it will be appreciated that a spinal fluid sampling system may be incorporated into the back of the chair 56.

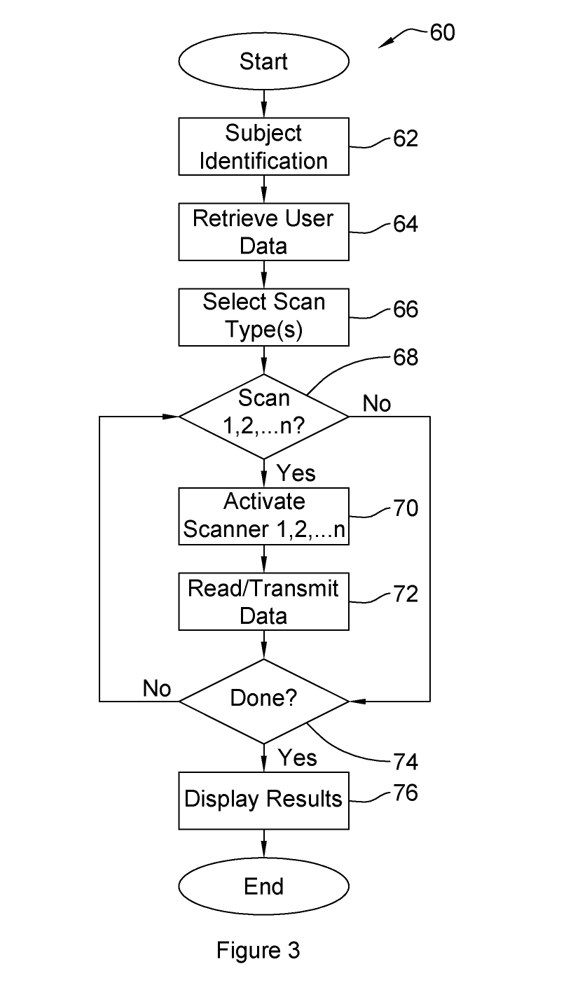

Referring now to FIG. 3, a flowchart depicting the process of scanning and displaying at least a portion of the human body utilizing the present system is shown generally at 60. The process begins with subject identification in step 62, by accessing or creating a user entry in the database 12. If the user is existing within the database 12, the system will retrieve the user data in step 64 from the database 12, otherwise a new entry will be created therein. Prior to the commencement of scanning, the user may select the scan type(s) in step 66 through the user interface device 18. It will be appreciated that the method of permitting a user to identify the subject and select the scan type may be by any conventional means, such as, by way of non-limiting example, pull-down menus, check boxes, or typed entries on the user interface device 18. It will also be appreciated that any number and resolutions of the input scanners 28 may be provided depending upon the intended purpose. By way of non-limiting example, lower resolution input scanners may be used for causal locations, such as, by way of non-limiting example, gym or drug store where the user is looking to quickly check their status for information purposes only. It will also be appreciated that in such locations, less input scanners may also be utilized. Similarly, in more advanced locations such as doctors' offices or clinics, higher resolution scanners may be utilized as well as a greater number and variety thereof may be utilized so as to be useful as a diagnostic tool.

Once the scan types have been inputted by the user in step 66, the processor 14 will check to determine if an input scanner 28 has been selected to engage in step 68. If an input scanner 28 has been selected, it will be activated to scan in step 70, with the data read by the input scanner 28 and transmitted to the processing circuit in step 72. Step 74 verifies if all scans have been completed, repeating the process of steps 68 to 74 until the completion of all selected scans. It will be appreciated that the scans may occur simultaneously or sequentially. When all scans have been completed, the results are displayed in step 76 on the output displays 30, which may include a digital monitor 52 or a projection camera 54 producing a 3-dimensional holographic image, as set out above.

By analysing the data collected through the user interface device 18 and the input scanners 28, the processor 14 may determine the overall health of a user and predict future health risks. Over an extended timeline, the user may perform several scans, enabling the system to provide more accurate data and health predictions.

While specific embodiments of the invention have been described and illustrated, such embodiments should be considered illustrative of the invention only and not as limiting the invention as construed in accordance with the accompanying claims.

What is claimed is:

1. A system for scanning and displaying at least a portion of a body comprising:
at least one scanner operable to perform a scan and output a data signal pertaining to a body;
a processing circuit operable to receive said data signal from said at least one scanner and compile an image representing said body; and
a display operable to receive said image representing said body from said processing circuit and display said image to a user.

2. The system of claim 1 wherein said at least one scanner comprises a plurality of scanners.

3. The system of claim 2 wherein said image representing said body is compiled from said scan results from a plurality of scanners.

4. The system of claim 1 wherein said display comprises a screen.

5. The system of claim 1 wherein said display comprises a holographic projector.

6. The system of claim 1 wherein said image is displayed to a user in real time.

7. The system of claim 1 further comprising an outer casing having an interior therein operable to receive a user.

8. The system of claim 7 wherein said body comprises a body of said user located within said interior of said casing.

9. The system of claim 8 wherein said display is operable to display said image representing said body to said user within said interior of said casing.

10. A method for scanning and displaying at least a portion of a body comprising:
utilizing at least one scanner, performing a scan of a body;
outputting from said at least one scanner, a data signal pertaining to said body;
receiving at a processing circuit said data signal from said at least one scanner;
compiling an image representing said body; and
displaying said image representing said body received from said processing circuit to a user.

11. The method of claim 10 further comprising utilizing a plurality of scanners.

12. The method of claim 11 wherein said image representing said body is compiled from said scan results from said plurality of scanners.

13. The method of claim 10 wherein said image is displayed to a user in real time.

14. An apparatus for scanning and displaying at least a portion of a body comprising:
an outer casing having an interior therein operable to receive a user;
at least one scanner operable to perform a scan and output a data signal pertaining to a body of said user within said interior;
a processing circuit operable to receive said data signal from said at least one scanner and compile an image representing said body; and
a display operable to receive said image representing said body from said processing circuit and display said image to a user.

15. The apparatus of claim 14 wherein said at least one scanner comprises a plurality of scanning means.

16. The apparatus of claim 15 wherein said image representing said body is compiled from said scan results from a plurality of scanners.

17. The apparatus of claim 14 wherein said display comprises a screen.

18. The apparatus of claim 14 wherein said display comprises a holographic projector.

19. The apparatus of claim 14 wherein said image is displayed to a user in real time.
